(19) United States
(12) Patent Application Publication
(10) Pub. No.: US 2007/0082064 A1
(43) Pub. Date: Apr. 12, 2007

(54) Nutritional or dietary supplement for the treatment of macular degeneration

(76) Inventor: Paul L. Krawitz, Cold Spring Harbor, NY (US)

Correspondence Address: DILWORTH & BARRESE, LLP, 333 EARLE OVINGTON BLVD., SUITE 702, UNIONDALE, NY, 11553, US

(21) Appl. No.: 11/546,050

(22) Filed: Oct. 11, 2006

Related U.S. Application Data
(60) Provisional application No. 60/725,978, filed on Oct. 12, 2005.

(51) Int. Cl. A61K 36/45; A61K 36/68; A61K 31/385; A61K 33/34; A61K 36/87

(52) U.S. Cl. 424/638; 424/641; 424/732; 424/766; 514/474; 514/440; 424/738; 424/702; 514/690; 514/18; 514/763

(57) ABSTRACT

A nutritional or dietary supplement composition that promotes retinal health through the prevention and/or treatment of visual acuity loss by reducing the risk of developing age-related macular degeneration in persons with early age-related macular degeneration. The essential ingredients of the nutritional or dietary supplement composition are vitamin C, micronutrients and other antioxidants. The essential ingredients are provided in an oral dosage. The composition is taken in the form of one or two tablets or capsules daily.

### CROSS REFERENCE TO RELATED APPLICATION

This application claims the benefit under 35 U.S.C. §119 (e) to Provisional Application No. 60/725,978, filed Oct. 12, 2005 and entitled “NUTRITIONAL SUPPLEMENT FOR THE TREATMENT OF MACULAR DEGENERATION,” the contents of which are incorporated by reference herein.

### FIELD OF THE INVENTION

The present invention relates to a nutritional or dietary supplement that promotes retinal health through the prevention and/or treatment of visual acuity loss by reducing the risk of developing age-related macular degeneration. More specifically, the present invention relates to a high-dosage zinc and antioxidant nutritional or dietary supplement composition, methods of administering the inventive composition for treatment and prevention of symptoms of age-related macular degeneration of the eye and a method of manufacturing a nutritional composition.

### BACKGROUND OF THE INVENTION

Dietary supplements are taken for a variety of reasons including the improvement of vision or prophylaxis of vision loss. Dietary supplements are generally in the form of powders, tablets, capsules or gel-caps and comprise a variety of vitamins, minerals, and herbal or other organic constituents.

Macular degeneration is a medical term that applies to any of several disease syndromes which involve a gradual loss or impairment of eyesight due to cell and tissue degeneration of the yellow macular region in the center of the retina. Age-related macular degeneration (AMD) is the most common form of this type of disease. AMD affects millions of Americans over the age of 60, and is the leading cause of new blindness among the elderly. It is characterized and usually diagnosed by the presence of elevated levels of two types of cellular debris within the retina, called drusen and lipofuscin. These types of cellular debris may accumulate to abnormal levels for a number of reasons, including: (1) retinal cell damage caused by repeated exposure to too much light; (2) inherited genetic factors; (3) poor overall health of an individual; and (4) insufficient quantities of anti-oxidant compounds. Accumulation of drusen occurs within the capillaries and in the Bruch's membrane, and can impede the transport of oxygen and nutrients to the retinal tissues, and the removal of metabolic wastes from the tissues. Accumulations of lipofuscin occurs within a cellular layer which underlies the photoreceptors and which is responsible for nourishing, replenishing and removing wastes from these highly active visual cells. Accumulation of one or both of these types of debris can disrupt the normal metabolic and cellular processes which must occur in order to maintain retinal and visual health.

Attention has been focused on the possible involvement of various vitamins and minerals in retinal disease. Vitamin E is a well-known antioxidant that has been reported to work synergistically with vitamin C in protecting vital cell function from normal oxidants. Supplement formulations providing total daily dosages from 400 IU to 500 IU have been promoted for the treatment and prophylaxis of eye diseases. This daily dosage of vitamin E is equivalent to approximately 13 to 18 times the U.S. recommended daily allowance (RDA) for vitamin E. Once ingested, vitamin E is stored within the body and can contribute to the total body pool of vitamin E for up to one year.

Results from a Johns Hopkins University analysis of clinical trial data suggests that taking daily doses of vitamin E that exceed 400 international units (IU) can increase the risk of death. The researchers arrived at their conclusion after analyzing death rates in 19 unrelated clinical trials that studied vitamin E supplementation for various health conditions. The trials took place from 1993 to 2004 and all together included more than 136,000 patients in North America, Europe and China. The vitamin E doses used in the different trials ranged from 15 to 2000 IU per day; the average daily intake was about 400 IU. The study used a technique called meta-analysis to arrive at the results that were presented on Nov. 10, 2004, at the American Heart Association's Scientific Sessions in New Orleans.

Prior to the concerns raised by this study, vitamin E had previously no known toxicity, except for its anticoagulant effect. The results of the Hopkins' analysis found that taking a daily dose of 200 IU per day presented no increased risk of death and suggested that it might benefit health. However, the researchers found an increased risk of death at daily doses exceeding 200 IU; for those taking daily doses of 400 IU or more the risk of death was about 10 percent higher than among those taking placebos.

Other issues concerning vitamin E include the recognition in scientific literature that vitamin E does not substantially decrease mortality from heart disease in patients with known coronary artery disease, may in some cases increase the risk of heart attack, and there have been reported cases of enhanced anticoagulant effect in patients taking oral anticoagulants.

Other supplements, such as carotene, (a dimer of vitamin A that comes in two forms α and β-carotene) used to treat retinal disease may posses undesirable effects. For example, beta-carotene has been shown in a number of large population studies to raise the risk of lung cancer in patients who smoke.

Presently treatment for macular degeneration includes early diagnosis and careful follow-up with protection against exposure to ultraviolet light and prescribed dosages of anti-oxidant vitamins and zinc.

If a treatment modality could slow down the progression of macular degeneration, with out the potential risks for side effects from certain nutritional and vitamin supplements (e.g. vitamin E and beta-carotene) it would have a tremendous impact on the large number of individuals who suffer this disease.

A need therefore still exists in the art to provide improved methods and compositions that are vitamin E and/or beta-carotene free for the treatment of macular degeneration.

### SUMMARY OF THE INVENTION

The present invention provides an orally administered composition for the treatment of age-related macular degeneration, said composition comprising an effective macular degeneration treatment amount of a mixture of:
a) vitamin C;
b) lutein;
c) alpha-lipoic acid;
d) zeaxanthin;
e) bilberry;
f) grapeseed extract;
g) zinc; and
at least one micronutrient selected from the group consisting of vitamin B-6, selenium, copper, and L-gluthione.

The present invention provides a method of treating age-related macular degeneration of the eye in a host, said method comprising orally administering to said host a composition comprising an effective macular degeneration treatment amount of a mixture of:
a) vitamin C;
b) lutein;
c) alpha-lipoic acid;
d) zeaxanthin;
e) bilberry;
f) grapeseed extract;
g) zinc; and
at least one micronutrient selected from the group consisting of vitamin B-6, selenium, copper, and L-gluthione.

The present invention provides a method of preventing age-related macular degeneration of the eye in a host, said method comprising orally administering to said host a composition comprising an effective macular degeneration prevention amount of a mixture of:
a) vitamin C;
b) lutein;
c) alpha-lipoic acid;
d) zeaxanthin;
e) bilberry;
f) grapeseed extract;
g) zinc; and
at least one micronutrient selected from the group consisting of vitamin B-6, selenium, copper, and L-gluthione.

The present invention provides a method of manufacturing a nutritional or dietary supplement composition comprising high-dose zinc, antioxidants, and other micronutrients effective in the prevention and treatment of macular degeneration and/or visual acuity loss comprising blending together each of the ingredients of the composition of the present invention.

The present invention also provides for a method of strengthening and promoting retinal health for the safe and effective prevention and/or treatment visual acuity loss.

### DETAILED DESCRIPTION OF THE INVENTION

The invention is based on the discovery that macular degeneration can be effectively treated and even prevented by administering effective amounts of antioxidants and micronutrients ingredients in the substantial absence of vitamin E and/or beta-carotene. To this end, the invention provides a method for treating macular degeneration by administration of an effective amount of vitamin C; lutein; alpha-lipoic acid; zeaxanthin; bilberry; grapeseed extract; zinc; and at least one micronutrient selected from the group consisting of vitamin B-6, selenium, copper, L-gluthione and mixture thereof to a human in need of such treatment.

The method of manufacturing the composition of the present invention includes procedures that one skilled in the art of Good Manufacturing Procedure (GMP) and production of high quality pharmaceutical formulations would use. The method comprises blending together each of the following ingredients: vitamin C; vitamin B-6; zinc; selenium; copper; lutein; zeaxanthin; bilberry; grapeseed extract; alpha-lipoic acid; and 5 mg L-glutathione into a suitable dosage form and in appropriate quantities. The method includes the use of know and conventional manufacturing excipients, e.g., flavorants, preservatives, stabilizers, and the like.

In one embodiment of the present invention, the preparation is preferably administered as a capsule twice daily with meals. However, it should be understood that the word “composition,” “preparation” or “formulation” as used herein is intended to refer collectively to these ingredients (i.e., components or compounds) and amounts whether taken separately by a patient or whether included in a single capsule or other ingestible medium. Suitable dosage forms include all dosage forms know in the art, such as, for example, capsules, tablets, liquids, sublingual forms and the like.

The terms “ingredient,” “component” and “compound,” as understood herein, refer to the pharmacologically active antioxidants and micronutrients and substances that comprise the composition of the invention.

The phrase “effective amount” is used throughout the specification to describe concentrations or amounts of the component ingredients according to the present invention which may be used to produce a favorable change in the symptomology, disease or condition treated, whether that change is a decrease in or reversal of the effects of symptomology or disease state depending upon the disease state or condition treated. In the present invention, in preferred aspects, an effective amount is that amount which is used to treat the symptomology associated with macular degeneration. The total daily effective macular degeneration treatment/prevention (and/or retinal health promoting) amount can be administered in one capsule or dosage form, or preferably in divided doses or multiple capsules, which in total deliver the effective amount of the composition of the present invention.

Vitamin C is an antioxidant that is found in high concentrations both in the normal human lens and in the aqueous humor that surrounds the lens. Vitamin C concentrations decrease with age and, in particular, in patients who have senile cataract. Increasing dietary Vitamin C generally increases the concentration of ascorbate in the aqueous humor and in the human lens. There is no known optimal daily dose of Vitamin C, although the U.S. recommended daily allowance (RDA) is 60 mg. However, dosages of 2.0 grams and more have frequently been taken as a supplement for general health. In one embodiment of the present invention, Vitamin C is present in the composition in the form of sodium ascorbate because of it being easily dissolved in the digestive system and causing relatively minimal irritation. In another embodiment of the present invention, Vitamin C is present in the composition in the amount of about 100 mg to about 1000 mg. In yet another embodiment Vitamin C is present in the composition in the amount of about 500 mg, which is the preferred total dosage of about 0.5 grams/day.

Vitamin B-6 in the form pyridoxine hydrochloride is one of a group of naturally occurring pyridines that is metabolically and functionally interrelated to pyridoxal, and pyridoxamine. Vitamin B-6 major functions are most closely related to protein and amino acid metabolism. The vitamin is a part of the molecular configuration of many enzymes (a coenzyme), notably glycogen phosphorylase, various transaminases, decarboxylases, and deaminases. The latter three are essential for the anabolism and catabolism of proteins. Pyridoxine also aids in fat and carbohydrate metabolism; aids in the formation of antibodies; maintains the central nervous system; aids in the removal of excess fluid of premenstrual women; promotes healthy skin; reduces muscle spasms, leg cramps, hand numbness, nausea and stiffness of hands; and helps maintain a proper balance of sodium and phosphorous in the body.

In one embodiment of the invention, vitamin B-6 is present in the composition in an amount from about 1 mg to about 40 mg, and in another embodiment, vitamin B-6 is present in the composition in the amount of about 20 mg.

Zinc is known to be important to the health of the retina and has been shown to be significantly better than placebo in slowing macular degeneration changes. Zinc is also known to be an important cofactor for a whole multitude of metalloenzymes, not the least of which is superoxide dismutase, which scavenges the potent oxidizer-superoxide. Dosages of about 10 mg to 150 mg of zinc a day have been known in the past to be well tolerated without difficulty. The U.S. recommended daily allowance (RDA) for zinc is 15 mg. While other salt forms such as acetate, sulfate, picolinate, phosphate, and gluconate can be used, in a preferred embodiment of the invention, the composition contains zinc in the form of zinc oxide, because of it being readily dissolved, causing minimal irritation, and effecting rapid, complete, and satisfactory plasma zinc content. In one embodiment, zinc is present in the composition in an amount from about 10 mg to about 150 mg of zinc in the form of zinc oxide. In another embodiment of the invention, zinc is present in the composition in an amount of about 80 mg in the form of zinc oxide.

Copper is another important cofactor for metalloenzymes, and is a second necessary cofactor for superoxide dismutase. Copper has been shown to decrease in individuals over 70 years of age, and if copper is significantly decreased, superoxide dismutase has been shown to have decreased function. Copper is also protective of zinc toxicity, which blocks some of the zinc absorption and, therefore, decreases bioavailability.

The U.S. RDA for copper is 2 mg. 2-3 mg of copper per day has been estimated to be safe and provide adequate daily dietary intake. Some copper absorption will be blocked by the 80 mg of daily zinc as provided above. As a result, the present composition preferably utilizes about 2 mg/day. In one embodiment of the present invention, copper is provided in the form of copper oxide in an amount of about 0.5 mg to about 4 mg, and preferably in an amount of about 2 mg.

Selenium is a necessary cofactor for metalloenzymes, particularly GSHPx, which scavenges peroxides. Significantly, it has been shown that macular degeneration is inversely related with plasma activity of GSHPx and suggested that its activity is an indication of the adequacy of selenium nutritional status. There is no U.S. RDA for selenium. However, safe and adequate daily dietary intake of selenium is about 50 to 200 micrograms (mcg) for an adult. In one embodiment of the present invention, selenium is present in the composition in an amount from about 100 mcg to about 300 mcg. In another embodiment of the present invention, selenium is present in an amount of about 200 mcg.

Lutein is a xanthophyll found in high concentrations in the macula of the eye and in the central part of the retina. It serves important roles in vision to help filter ultraviolet wavelengths of light to prevent damage to the eye lens and macula. Lutein's antioxidant properties are believed to help protect the macula, which is rich in polyunsaturated fats, from light-induced free radicals. Lutein cannot be produced by the body, and consequently, must be ingested. Thus, lutein has become increasingly used in nutritional supplements for the prevention and/or treatment of vision losses due to macular degeneration, cataracts and retinitis pigmentosa.

Lutein has been shown to have significant potential in the prevention of age-related macular degeneration (AMD), and helps build macular pigment density, a critical factor in the health of the macula and the retina. Further, it has been shown that the accumulation of lutein in the macular pigment is dependent upon dietary intake and that the density of the macular pigment is related to the preservation of visual sensitivity and protection from. Other vision loss problems, such as cataracts and retinitis pigmentosa may also be stopped or reduced with a high intake of lutein.

In one embodiment of the invention, lutein is present in the composition in an amount of from about 1 mg to about 40 mg, and in another embodiment, lutein is present in the composition in the amount of about 20 mg.

Zeaxanthin, like lutein, is a major carotenoid that makes up the macular pigment of the eye's retina, and its antioxidant properties protect the eye from light-induced damage and macular degeneration. In one embodiment of the present invention, zeaxanthin is present in the composition in an amount from about 1 mg to about 10 mg. In a more preferred embodiment of the invention, zeaxanthin is present in the composition in an amount of about 4.75 mg.

Bilberry (vaccinium myrtil) is a rich source of anthocyanosides, which are useful antioxidants. Studies have shown that Bilberry has a significant positive effect on night vision and can reduce or reverse effects of degenerative eye disorders such as macular degeneration. Bilberry may have other beneficial effects on capillaries due to the strong antioxidant properties of its anthocyanidin bioflavonoids. In one embodiment of the present invention, bilberry is present in the composition in an amount of about 10 mg to about 200 mg. In a preferred embodiment of the invention, bilberry is present in the composition in an amount of about 50 mg.

Grapeseed extract contains natural bioflavonoids called proanthocyanidins which help strengthen and protect cell membranes from oxidative damage caused by free radicals. Studies have found that supplementation with grapeseed extract substantially increased levels of antioxidants in the blood. In one embodiment of the present invention, grapeseed extract is present in the composition in an amount of about 1 mg to about 40 mg. In another embodiment, grapeseed extract is present in the composition in the amount of about 20 mg.

Glutathione is a protein composed of three amino acids, glycine, glutamic acid and cysteine. High levels of glutathione are found in the lens of the eye. Glutathione is also effective in preventing macular degeneration. In one embodiment of the present invention, L-glutathione is present in the composition in an amount of about 1 mg to about 100 mg. In a preferred embodiment of the invention, L-glutathione is present in the composition in an amount of about 10 mg.

Alpha-lipoic acid provides superior antioxidant protection due to the fact that it enhances the potency of other antioxidants in the body. In one embodiment of the present invention, alpha-lipoic acid is present in the composition in an amount of about 10 mg to about 30 mg. In a preferred embodiment of the invention, alpha-lipoic acid is present in the composition in an amount of about 20 mg.

Typically the composition of the present invention may include pharmaceutically acceptable components such as lactose, glucose, sucrose, corn starch, potato starch, cellulose esters such as cellulose acetate, ethyl cellulose, magnesium stearate, calcium silicate, precipitated silica, talc, fatty acids such as stearic acid, microcrystalline cellulose, carnauba wax and the like. Diluents and other additives such as one or more pharmaceutically acceptable binding agents, fillers, supports, thickening agents, taste-improving agents, coloring agents, preservatives, stabilizers, regulators, emulsifiers, flow agents, absorbents, and the like or mixtures thereof may be used depending on the form of the composition employed.

The formulation of the invention can be prepared by standard techniques known in the art. As appreciated by the skilled artisan, the desired processing technique will vary depending upon the exact types and amounts of ingredients present, processing temperature, and the like.

In the formulation of the present invention lutein and zeaxanthin are protected from degradation during periods of storage prior to ingestion. To increase the stability of the lutein and zeaxanthin compounds to oxidative degradation, it is advantageous to add stabilizers such as alpha-tocopherol, butylated hydroxytoluene, butylated hydroxyanisole, ascorbic acid or ethoxyquin to the manufacturing process. An antioxidant system, e.g. “beadlet,” may be used to protect the lutein and zeaxanthin compounds. In the beadlet process, lutein and zeaxanthin may be blended with a non-pharmacologically effective amounts of synthetic vitamin E (e.g. dl-alpha-tocopherol). If dl-alpha-tocopherol is used to stabilize lutein and zeaxanthin, sub-therapeutic quantities of synthetic vitamin E may be present in the formulation. As such, synthetic vitamin E would be present in amounts exhibiting bioactivity that would be equivalent to about 2.25 IU of the natural form of vitamin E per daily dosage. At this concentration in the formulation the amount of synthetic vitamin E is insufficient to cause any of the side effects described supra.

The formulation of the invention may also contain flavorants such as fruit and/or other similar flavors, caramel, and the like. When present, flavorants are typically present in an amount of about 0.005 to about 0.3 mg/ml, more typically about 0.05 to about 0.1 mg/ml.

The formulation of the invention optionally can also contain other ingredients such as preservatives (e.g., sodium benzoate, methyl paraben, ethyl paraben, propyl paraben, and the like), stabilizers (e.g., ferric ammonium citrate, ferrous sulfate, and the like), etc.

Having described the invention in detail, it will be apparent that numerous modifications and variations are possible.

The following examples are offered only to illustrate the invention, and should not be interpreted as a limitation thereon.

### EXAMPLE 1

A composition for the treatment of age-related macular degeneration comprising the following:
vitamin C 500 mg
lutein 20 mg
alpha-lipoic acid 20 mg
zeaxanthin 4.75 mg
bilberry 50 mg
grapeseed extract 20 mg
zinc 80 mg
vitamin B-6 20 mg
selenium 200 mcg
copper 2 mg
L-gluthione 10 mg

The composition of Example 1 provides a safe and effective composition that strengthens and promotes retinal health and the treatment of age-related macular degeneration of the eye in a patient in need of such treatment.

Obviously, other modifications and variations of the present invention are possible in light of the above teachings. It is, therefore, to be understood that changes may be made in the particular embodiments described above which run within the full intended scope of the invention.

1. An orally administered composition for the treatment of age-related macular degeneration, said composition comprising an effective macular degeneration treatment amount of a mixture of:
a) vitamin C;
b) lutein;
c) alpha-lipoic acid;
d) zeaxanthin;
e) bilberry;
f) grapeseed extract;
g) zinc; and
at least one micronutrient selected from the group consisting of vitamin B-6, selenium, copper, and L-gluthione.

2. The composition of claim 1 wherein the vitamin C is provided in the form of ascorbic acid, the zinc is provided in the form of zinc oxide, the vitamin B-6 is provided in the form of pyridoxine hydrochloride, and the copper is provided in the form of cupric oxide.

3. The composition of claim 1 wherein the vitamin C is present in an amount from about 250 mg to about 700 mg, the zinc is present in an amount from about 10 mg to about 150 mg, the copper is present in the amount from about 0.5 mg to about 4 mg, the alpha-lipoic acid is present in the amount from about 10 mg to about 30 mg, the lutein is present in the amount from about 1 mg to about 40 mg, the bilberry is present in the amount from about 10 mg to about 200 mg, the grapeseed extract is present in the amount from about 1 mg to about 40 mg, the L-glutathione is present in the amount from about 1 mg to about 100 mg, the zeaxanthin is present in the amount from about 1 mg to about 10 mg, the vitamin B-6 is present in the amount from about 1 mg to about 40 mg, and the selenium is present in the amount from about 100 mcg to about 300 mcg.

4. The composition of claim 3 wherein the vitamin C is present in an amount of about 500 mg, the zinc is present in the amount of about 80 mg, the copper is present in the amount about 2 mg, the alpha-lipoic acid is present in the amount of about 20 mg, the lutein is present in the amount of about 20 mg, the bilberry is present in the amount of about 50 mg, the grapeseed extract is present in the amount of about 20 mg, the L-glutathione is present in the amount of about 10 mg, the zeaxanthin is present in the amount of about 4.75 mg, the vitamin B-6 is present in the amount of about 20 mg, and the selenium is present in the amount from about 200 mcg.

5. The composition of claim 1 wherein the composition is in the form of a capsule for daily oral ingestion by a human.

6. The composition of claim 1 wherein the composition is in the form of more than one capsule for daily oral ingestion by a human.

7. A method of strengthening and promoting retinal health in a host, said method comprising administering orally to said host, on a daily basis, an effective retinal health promoting amount of the composition of claim 1.

8. The method of claim 7 wherein the vitamin C is provided in the form of ascorbic acid, the zinc is provided in the form of zinc oxide, the vitamin B-6 is provided in the form of pyridoxine hydrochloride, and the copper is provided in the form of cupric oxide.

9. The method of claim 7 wherein the vitamin C is present in an amount from about 250 mg to about 700 mg, the zinc is present in an amount from about 10 mg to about 150 mg, the copper is present in the amount from about 0.5 mg to about 4 mg, the alpha-lipoic acid is present in the amount from about 10 mg to about 30 mg, the lutein is present in the amount from about 1 mg to about 40 mg, the bilberry is present in the amount from about 10 mg to about 200 mg, the grapeseed extract is present in the amount from about 1 mg to about 40 mg, the L-glutathione is present in the amount from about 1 mg to about 100 mg, the zeaxanthin is present in the amount from about 1 mg to about 10 mg, the vitamin B-6 is present in the amount from about 1 mg to about 40 mg, and the selenium is present in the amount from about 100 mcg to about 300 mcg.

10. The method of claim 9 wherein the vitamin C is present in an amount of about 500 mg, the zinc is present in the amount of about 80 mg, the copper is present in the amount about 2 mg, the alpha-lipoic acid is present in the amount of about 20 mg, the lutein is present in the amount of about 20 mg, the bilberry is present in the amount of about 50 mg, the grapeseed extract is present in the amount of about 20 mg, the L-glutathione is present in the amount of about 10 mg, the zeaxanthin is present in the amount of about 4.75 mg, the vitamin B-6 is present in the amount of about 20 mg, and the selenium is present in the amount of about 200 mcg.

11. A method of treating age-related macular degeneration of the eye in a host, said method comprising administering orally to said host a composition comprising an effective macular degeneration treatment amount of a mixture of:
a) vitamin C;
b) lutein;
c) alpha-lipoic acid;
d) zeaxanthin;
e) bilberry;
f) grapeseed extract;
g) zinc; and
at least one micronutrient selected from the group consisting of vitamin B-6, selenium, copper, and L-gluthione.

12. The method of claim 11 wherein the vitamin C is provided in the form of ascorbic acid, the zinc is provided in the form of zinc oxide, the vitamin B-6 is provided in the form of pyridoxine hydrochloride, and the copper is provided in the form of cupric oxide.

13. The method of claim 11 wherein the vitamin C is present in an amount from about 250 mg to about 700 mg, the zinc is present in an amount from about 10 mg to about 150 mg, the copper is present in the amount from about 0.5 mg to about 4 mg, the alpha-lipoic acid is present in the amount from about 10 mg to about 30 mg, the lutein is present in the amount from about 1 mg to about 40 mg, the bilberry is present in the amount from about 10 mg to about 200 mg, the grapeseed extract is present in the amount from about 1 mg to about 40 mg, the L-glutathione is present in the amount from about 1 mg to about 100 mg, the zeaxanthin is present in the amount from about 1 mg to about 10 mg, the vitamin B-6 is present in the amount from about 1 mg to about 40 mg, and the selenium is present in the amount from about 100 mcg to about 300 mcg.

14. The method of claim 13 wherein the vitamin C is present in an amount of about 500 mg, the zinc is present in the amount of about 80 mg, the copper is present in the amount of about 2 mg, the alpha-lipoic acid is present in the amount of about 20 mg, the lutein is present in the amount of about 20 mg, the bilberry is present in the amount of about 50 mg, the grapeseed extract is present in the amount of about 20 mg, the L-glutathione is present in the amount of about 10 mg, the zeaxanthin is present in the amount of about 4.75 mg, the vitamin B-6 is present in the amount of about 20 mg, and the selenium is present in the amount of about 200 mcg.

15. A method of preventing age-related macular degeneration of the eye in a host, said method comprising administering orally to said host a composition comprising an effective macular degeneration prevention amount of a mixture of:
a) vitamin C;
b) lutein;
c) alpha-lipoic acid;
d) zeaxanthin;
e) bilberry;
f) grapeseed extract;
g) zinc; and
at least one micronutrient selected from the group consisting of vitamin B-6, selenium, copper, and L-gluthione.

16. The method of claim 15 wherein the vitamin C is provided in the form of ascorbic acid, the zinc is provided in the form of zinc oxide, the vitamin B-6 is provided in the form of pyridoxine hydrochloride, and the copper is provided in the form of cupric oxide.

17. The method of claim 15 wherein the vitamin C is present in an amount from about 250 mg to about 700 mg, the zinc is present in an amount from about 10 mg to about 150 mg, the copper is present in the amount from about 0.5 mg to about 4 mg, the alpha-lipoic acid is present in the amount from about 10 mg to about 30 mg, the lutein is present in the amount from about 1 mg to about 40 mg, the bilberry is present in the amount from about 10 mg to about 200 mg, the grapeseed extract is present in the amount from about 1 mg to about 40 mg, the L-glutathione is present in the amount from about 1 mg to about 100 mg, the zeaxanthin is present in the amount from about 1 mg to about 10 mg, the vitamin B-6 is present in the amount from about 1 mg to about 40 mg, and the selenium is present in the amount from about 100 mcg to about 300 mcg.

18. The method of claim 17 wherein the vitamin C is present in an amount of about 500 mg, the zinc is present in the amount of about 80 mg, the copper is present in the amount about 2 mg, the alpha-lipoic acid is present in the amount of about 20 mg, the lutein is present in the amount of about 20 mg, the bilberry is present in the amount of about 50 mg, the grapeseed extract is present in the amount of about 20 mg, the L-glutathione is present in the amount of about 10 mg, the zeaxanthin is present in the amount of about 4.75 mg, the vitamin B-6 is present in the amount of about 20 mg, and the selenium is present in the amount of about 200 mcg.

19. A method of manufacturing a composition comprising blending together each of the components recited in the composition of claim 1.
